(19) United States
(12) Patent Application Publication
(10) Pub. No.: US 2024/0075044 A1
(43) Pub. Date: Mar. 7, 2024

(54) CO-PROCESSED PRE-FORMULATED EXCIPIENT COMPOSITION FOR POORLY SOLUBLE ACTIVE PHARMACEUTICAL INGREDIENTS

(71) Applicant: Sigachi Industries Limited, Hyderabad (IN)

(72) Inventors: Amit Raj Sinha, Hyderabad (IN); Monika Tomar, Bharuch (IN); Rauf Pathan, Bharuch (IN)

(73) Assignee: Sigachi Industries Limited, Hyderabad (IN)

(21) Appl. No.: 18/241,260

(22) Filed: Sep. 1, 2023

(30) Foreign Application Priority Data
Sep. 1, 2022 (IN) 202241050056

(51) Int. Cl. A61K 31/549; A61K 9/20

(52) U.S. Cl. CPC A61K 31/549; A61K 9/2009; A61K 9/2013; A61K 9/2054

(57) ABSTRACT

The present disclosure pertains to a particulate co-processed pre-formulated excipient composition and methods for producing the same. In particular, the present disclosure provides a particulate co-processed pre-formulated excipient composition for enhancing dissolution rate of poorly soluble active pharmaceutical ingredient, wherein said excipient composition comprises a co-processed mixture of a filler-binder, a glidant, a disintegrant, a solubility enhancer, and a lubricant. The co-processed pre-formulated excipient composition has higher bulk density, bigger particle size, excellent flowability, good physical properties of excipient delivered good quality of tablet in terms tablet hardness, disintegration time and dissolution rate.

### CROSS-REFERENCE TO RELATED APPLICATION

The present application claims priority to and the benefit from Indian Patent Application No. 202241050056 filed on Sep. 1, 2022, the contents of which are incorporated herein in its entirety.

### FIELD OF THE INVENTION

The present disclosure pertains to a particulate co-processed pre-formulated excipient composition and methods for producing the same. In particular, the present disclosure provides a particulate co-processed pre-formulated excipient composition for enhancing dissolution rate of poorly soluble active pharmaceutical ingredients (APIs), wherein said excipient composition comprises a co-processed mixture of a filler-binder, a glidant, a disintegrant, a solubility enhancer, and a lubricant.

### BACKGROUND OF THE INVENTION

The background description includes information that may be useful in understanding the present invention. It is not an admission that any of the information provided herein is prior art or relevant to the present invention, or that any publication specifically or implicitly referenced is prior art.

A list of excipients is used to manufacture various dosage forms. Generally, dosage forms divided into three types viz., solid, liquid, and semi-solid dosage forms. Solid dosage forms may be Tablet, Capsules, and powders. Tablet is most popular and better patient compliance, there are three basically methods are used to manufactured tablets, like wet granulation, dry granulation, and direct compression. In wet granulation method required to manufacture granules after that compress tablets, whereas manufacturing of granulations is time consuming process, and it is Costlier method. But direct compression method is fast method for tablet manufacturing.

As a result of their numerous advantages, such as the high physical and chemical stability, the precision of dosage, the ease of administration, portability, cheap mass production, and the ability to provide a variety of release patterns of active ingredients, tablets are the most commonly used form of delivering active pharmaceutical ingredients on site to cure disease. Tablet can be obtained by compressing powders formulated in an appropriate manner. Tablets are manufactured by three ways in the pharmaceutical industry: wet granulation, dry granulation, and direct compression (DC). Granulation techniques involve multiple steps and manufacturing challenges, leading to a substantial increase in the cost and time of production. Direct tableting is the preferred method.

DC tableting as a technique involves the compression of a dry blend of powders that comprises drugs and various excipients, which results in a number of benefits including time and cost savings. With this formulation procedure, granulation is no longer necessary before tableting. In a word, the simplicity and cost-effectiveness of DC have positioned it as a preferred alternative. However, DC process is highly influenced by powder characteristics such as flowability and compressibility. At the beginning, untreated excipients were applied for DC, such as powder cellulose or α-lactose-monohydrate. Then, the excipients were improved by using various manufacturing processes, such as spray drying (spray-dried lactose), or by using a special sieve fraction, but the improvement is limited and does not always provide the requisite performance for drugs in formulation and manufacture. Co-processed pre-formulated excipient is the next technological innovation for DC, which fulfils the increasing demand for multifunctional excipients for DC tableting. Co-processed pre-formulated excipients are prepared by incorporating one excipient into the particle structure of another excipient using processes such as co-drying, hot-melt extrusion, freeze thawing, and co-precipitation. Co-processing implies the combination of two or more established excipients in some common process (granulation, spray drying, milling, co-crystallization, etc.) in order to synergistically improve excipient functional properties and mask undesired properties. This concept is based on component interactions on the sub particle level, wherein particles of one excipient can be incorporated on the surface or within the core of particles of another excipient. Since no chemical changes occur during co-processing, the obtained product can be considered as a mixture of the existing excipient and thus is not subjected to extensive toxicological studies The co-processed multifunctional excipients are introduced to achieve better characteristics and tableting properties, including high compatibility, high intrinsic flow, good lubricating efficiency, improved blending properties and good binding properties, than a single substance or the physical mixtures.

Traditional compressible mixtures are made by combining an API with appropriate excipient components such as diluents, fillers/carriers, binders or adhesives, disintegrants, glidants or flow promoters, colours, and flavourings, among others. Once the material has been thoroughly mixed, a lubricating excipient is added, and the material is compacted into a tablet. The mixture would next be compacted into tablets and combined with an API and, if desired, a lubricant. Prior to compression, a lubricant is combined with the carrier and active substance, regardless of the tableting technique.

Solubility, the phenomenon of dissolution of solute in solvent to give a homogenous system, is one of the important parameters to achieve desired concentration of drug in systemic circulation for desired pharmacological response. Low aqueous solubility is the major problem encountered with formulation development of new chemical entities as well as for the generic development. Solubility is not to be confused with the ability to dissolve or liquefy a substance, since these processes may occur not only because of dissolution but also because of a chemical reaction. For example, zinc is insoluble in hydrochloric acid, but does dissolve in it by chemically reacting into zinc chloride and hydrogen, where zinc chloride is soluble in hydrochloric acid. Solubility does not also depend on particle size or other kinetic factors; given enough time, even large particles will eventually dissolve. More than approximate 40% API's developed in pharmaceutical industry are practically insoluble in water. Solubility is a major challenge for formulator. Any drug to be absorbed must be present in the form of solution at the site of absorption. Various techniques are used for the enhancement of the solubility of poorly soluble drugs which include physical and chemical modifications of drug and other methods like particle size reduction, crystal engineering, salt formation, solid dispersion, use of surfactant, complexation, and etc. Selection of solubility improving method depends on drug property, site of absorption, and required dosage form characteristics.

However, there is a perpetual need to optimise the process for producing ready-to-use co-processed pre-formulated excipient compositions for poorly soluble drugs in the art.

Thus, the present invention relates to ready-to-use co-processed pre-formulated excipient compositions for poorly soluble that are useful for the formulation of a wide variety of drugs.

### OBJECTS OF THE INVENTION

Objects of the present invention are to provide a particulate co-processed pre-formulated excipient composition for oral solid dosage forms.

An object of the present invention is to provide a particulate co-processed pre-formulated excipient composition that is ready-to-use.

An object of the present invention is to provide a particulate co-processed pre-formulated excipient composition for enhancing dissolution rate of poorly soluble active pharmaceutical ingredients (APIs).

An object of the present invention is to provide a particulate co-processed pre-formulated excipient composition that has higher bulk density, bigger particle size, excellent flowability, good physical properties of excipient delivered good quality of tablet in terms tablet hardness, disintegration time and dissolution rate.

An object of the present invention is to provide a particulate co-processed pre-formulated excipient composition comprising a co-processed mixture of a filler-binder, a glidant, a disintegrant, a solubility enhancer, and a lubricant.

Another object of the present invention is to provide a method for producing the particulate co-processed pre-formulated excipient composition comprising a co-processed mixture of a filler-binder, a glidant, a disintegrant, a solubility enhancer, and a lubricant.

Another object of the present invention is to provide a particulate co-processed pre-formulated excipient composition for tableting by wet granulation, direct compression and/or dry granulation.

Yet another object of the present invention is to provide a tablet formulation comprising a poorly soluble API, the particulate co-processed pre-formulated excipient composition, and optionally one or more excipients.

### SUMMARY OF THE INVENTION

Aspects of the present invention pertain to a particulate co-processed pre-formulated excipient composition and methods for producing the same. In particular, the present disclosure provides a particulate co-processed pre-formulated excipient composition for enhancing dissolution rate of poorly soluble active pharmaceutical ingredient, wherein said excipient composition comprises a co-processed mixture of a filler-binder, a glidant, a disintegrant, a solubility enhancer, and a lubricant.

In some aspects, the present invention provides a particulate co-processed pre-formulated excipient composition for oral solid dosage forms.

In some aspects, the present invention provides a particulate co-processed pre-formulated excipient composition that is ready-to-use.

In an aspect, the present invention provides a particulate co-processed pre-formulated excipient composition for enhancing dissolution rate of poorly soluble active pharmaceutical ingredients (APIs).

In an aspect, the present invention provides a particulate co-processed pre-formulated excipient composition that has higher bulk density, bigger particle size, excellent flowability, good physical properties of excipient delivered good quality of tablet in terms tablet hardness, disintegration time and dissolution rate.

In an aspect, the present invention provides a particulate co-processed pre-formulated excipient composition comprising a co-processed mixture of a filler-binder, a glidant, a disintegrant, a solubility enhancer, and a lubricant.

In an embodiment, the present invention provides a particulate co-processed pre-formulated excipient composition comprises based on total weight of the composition:
filler-binder in an amount of 88.0% w/w to 97.8% w/w;
glidant in an amount of 0.50% w/w to 3.0% w/w;
disintegrant in an amount of 1.5% w/w to 6.0% w/w;
solubility enhancer in an amount of 0.1% w/w to 1.0% w/w; and
lubricant in an amount of 0.1% w/w to 2.0% w/w.

In an embodiment, the present invention provides a particulate co-processed pre-formulated excipient composition comprises based on total weight of the composition:
MCC in an amount of 88.0% w/w to 97.8% w/w;
Colloidal silicon dioxide in an amount of 0.50% w/w to 3.0% w/w;
croscarmellose sodium in an amount of 1.5% w/w to 6.0% w/w;
sodium lauryl sulphate in an amount of 0.1% w/w to 1.0% w/w; and
magnesium stearate in an amount of 0.1% w/w to 2.0% w/w.

In another aspect, the present invention provides a method for producing the particulate co-processed pre-formulated excipient composition comprising a co-processed mixture of a filler-binder, a glidant, a disintegrant, a solubility enhancer, and a lubricant.

In an embodiment, the present invention provides a method for preparing the particulate co-processed pre-formulated excipient composition (FIG. 1), the method comprising the steps of:
a. forming a homogeneous slurry of filler-binder using demineralized water;
b. forming a homogeneous slurry of glidant using demineralized water;
c. forming a homogeneous slurry of disintegrant using demineralized water;
d. mixing the individual homogenous slurries of filler-binder, glidant and disintegrant by stirring to obtain a homogenous mixed slurry;
e. mixing a lubricant into the homogenous mixed slurry from step d), followed by proper mixing by stirring;
f. forming a homogeneous slurry of solubility enhancer using demineralized water;
g. adding the homogeneous slurry of solubility enhancer into the homogenous mixed slurry from step e), followed by proper mixing to obtain a slurry of co-processed pre-formulated excipient composition; and
h. spray or flash drying the slurry of co-processed pre-formulated excipient composition to obtain the particulate co-processed pre-formulated excipient composition; and
   wherein, the mixing is effected by agitation for 5 minutes at 25 rpm;
   wherein, temperature during mixing is maintained at 30° C. to 150° C.; and
   wherein, pH during mixing is maintained at 5 to 7.5.

In an embodiment, the present invention provides a method for preparing the particulate co-processed pre-formulated excipient composition (FIG. 1), the method comprising the steps of:
a. forming a homogeneous slurry of 88.0% w/w to 97.8% w/w MCC using demineralized water;
b. forming a homogeneous slurry of 0.50% w/w to 3.0% w/w colloidal silicon dioxide using demineralized water;
c. forming a homogeneous slurry of 1.5% w/w to 6.0% w/w croscarmellose sodium using demineralized water;
d. mixing the individual homogenous slurries of MCC, colloidal silicon dioxide and croscarmellose sodium by stirring to obtain a homogenous mixed slurry;
e. mixing 0.1% w/w to 2.0% w/w magnesium stearate powder into the homogenous mixed slurry from step d), followed by proper mixing by stirring;
f. forming a homogeneous slurry of 0.1% w/w to 1.0% w/w sodium lauryl sulphate using demineralized water;
g. adding the homogeneous slurry of 0.1% w/w to 1.0% w/w sodium lauryl sulphate into the homogenous mixed slurry from step e), followed by proper mixing to obtain a slurry of co-processed pre-formulated excipient composition; and
h. spray or flash drying the slurry of co-processed pre-formulated excipient composition to obtain the particulate co-processed pre-formulated excipient composition;
   wherein, the mixing is effected by agitation for 5 minutes at 25 rpm;
   wherein, temperature during mixing is maintained at 30° C. to 150° C.; and
   wherein, pH during mixing is maintained at 5 to 7.5.

In another aspect, the present invention provides a particulate co-processed pre-formulated excipient composition for tableting by wet granulation, direct compression and/or dry granulation.

In yet another aspect, the present invention provides a tablet formulation comprising a poorly soluble API, the particulate co-processed pre-formulated excipient composition, and optionally one or more excipients.

In a preferred embodiment, the present invention provides a tablet formulation comprising:
the particulate co-processed pre-formulated excipient composition in an amount ranging between 35% w/w and 65% w/w;
a poorly soluble active pharmaceutical ingredient in an amount ranging between 35% w/w and 65% w/w; and
optionally one or more excipients in an amount ranging between 1% w/w and 10% w/w.

Various objects, features, aspects and advantages of the inventive subject matter will become more apparent from the following detailed description of preferred embodiments.

### BRIEF DESCRIPTION OF DRAWINGS

Characteristics and advantages of the subject matter as disclosed in the present disclosure will become clearer from the detailed description of an embodiment thereof, with reference to the attached drawing, given purely by way of an example, in which:

FIG. 1 relates to the flow diagram of a method for preparing co-processed pre-formulated excipient composition

FIGS. 2A, 2B, and 2C relate to the scanning electron microscopic image co-processed pre-formulated excipient composition.

FIG. 3 relates to the API release profile of the co-processed pre-formulated excipient composition.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weight percentages, angles, and so forth, to describe and claim certain embodiments of the invention and are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Various terms as used herein are shown below. To the extent a term used in a claim is not defined below, it should be given the broadest definition persons in the pertinent art have given that term as reflected in printed publications and issued patents at the time of filing.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follows, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Various terms are used herein. To the extent a term used in a claim is not defined below, it should be given the broadest definition persons in the pertinent art have given that term as reflected in printed publications and issued patents at the time of filing.

The term “particulate co-processed pre-formulated excipient composition” as used herein, refers to a mixture of two or more components, i.e., a filler, a binder, a glidant and optionally lubricant and disintegrant, that have been co-processed using various means but not limited to co-milling, mechanical milling, spray drying, freeze-drying, and the like.

While a particular form of the invention has been illustrated and described, it will be apparent that various modifications can be made without departing from the spirit and scope of the invention.

The present disclosure pertains to a particulate co-processed pre-formulated excipient composition and methods for producing the same. In particular, the present disclosure provides a particulate co-processed pre-formulated excipient composition for enhancing dissolution rate of poorly soluble active pharmaceutical ingredient.

In an embodiment of the present invention, the particulate co-processed pre-formulated excipient composition has higher bulk density, bigger particle size, excellent flowability, better tableting profile, good physical properties of excipient delivered good quality of tablet in terms tablet hardness, disintegration time and dissolution rate.

In an embodiment of the present invention, the particulate co-processed pre-formulated excipient composition is a ready-to-use composition, which requires only the mixing of said excipient composition and API for tableting.

In some embodiments, the present invention provides a particulate co-processed pre-formulated excipient composition comprising a co-processed mixture of a filler-binder, a glidant, a disintegrant, a solubility enhancer, and a lubricant.

In an embodiment of the present invention, the filler-binder is selected from but not limited to starches, sugars, cellulose or modified cellulose such as microcrystalline cellulose (MCC), hydroxypropyl cellulose, lactose, or sugar alcohols like xylitol, lactitol, mannitol, sorbitol or maltitol. Mostr preferably MCC.

In an embodiment of the present invention, the glidant is selected from but not limited to silica gel, colloidal silica (colloidal silicon dioxide), fumed silica, precipitated silica, talc, and mixtures thereof. Most preferably colloidal silica (colloidal silicon dioxide).

In an embodiment of the present invention, the solubility enhancer is selected from but not limited to alkyl sulfates. Most preferably sodium lauryl sulphate.

In an embodiment of the present invention, the lubricant is selected from but not limited to magnesium stearate or sodium stearyl fumarate. Most preferably magnesium stearate.

In an embodiment of the present invention, the disintegrant is selected from the group consisting of croscarmellose sodium, sodium starch glycolate, crosspovidone or calcium carboxyl methyl cellulose. Most preferably croscarmellose sodium.

In an embodiment of the present invention, the term “poorly soluble API” refers to those API drugs having solubility range from 2 microgram to 2300 microgram/ml of aqueous solubility. Particularly, poorly soluble drugs include drugs selected from the group consisting of sparingly soluble, slightly soluble, very slightly soluble and practically insoluble drugs. For example Atorvastatin, Amilodipine, Atenolol, APAP, Enalapril, Acyclovir, Azithromycine, Carbamazepine, Clopidogrel, Indinavir, Rabeprazole, Stavudine, Venlafaxin, Cetirizine, Chlorpheniramine, Chlorpromazine, Ciprofloxacin, Diclofenac, Esomeprazole, Fexofenadine, Flucanozole, Tizanidine, Terbinafine, Trimethoprim, Valsartan, Ibuprofen, Divalproex, Metolazone, Methycarbamol, Losartan, Metoprolol, Metformin, Pantoprozole, Propranolol, Naproxen Na, Ranitidine, Raloxifene, Tagesrod, Efaverinz, Ezetimibe, Itraconazole, Ketoconazole, Loratidine, Lovastatin, Simvastatin, Metaxalone, and the like.

In some embodiments, the particulate co-processed pre-formulated excipient composition includes about 80% w/w to about 98 wt % of filler-binder; In some embodiments, about 80% w/w; about 80.5% w/w; about 81% w/w; about 81.5% w/w; about 82% w/w; about 82.5% w/w; about 83% w/w; about 83.5% w/w; about 84% w/w; about 84.5% w/w; about 85% w/w; about 85.5% w/w; about 86% w/w; about 86.5% w/w; about 87% w/w; about 87.5% w/w; about 88% w/w; about 88.5% w/w; about 89% w/w; about 89.5% w/w; about 90% w/w, about 90.5% w/w; about 91% w/w; about 91.5% w/w; about 92% w/w; about 92.5% w/w; about 93% w/w; about 93.5% w/w; about 94% w/w; about 94.5% w/w; about 95% w/w; about 95.5% w/w; about 96% w/w; about 96.5% w/w; about 97% w/w; about 97.5% w/w; about 97.6% w/w; about 97.7% w/w; about 97.8% w/w; about 97.9% w/w; or about 98% w/w. Most preferably 88.0% w/w to 97.8% w/w.

In some embodiments, the particulate co-processed pre-formulated excipient composition includes about 0.1% w/w to 5.0% w/w glidant; In some embodiments about 0.1% w/w; about 0.5% w/w; about 1% w/w; about 1.5% w/w; about 2% w/w; about 2.5% w/w; about 3% w/w; about 3.5% w/w; about 4% w/w; about 4.5% w/w; or about 5% w/w. Most preferably 0.50% w/w to 3.0% w/w.

In some embodiments, the particulate co-processed pre-formulated excipient composition includes about 0.1% w/w to 8.0% w/w disintegrant; In some embodiments about 0.1% w/w; about 0.5% w/w; about 1% w/w; about 1.5% w/w; about 2% w/w; about 3% w/w; about 4% w/w; about 5% w/w; about 6% w/w; about 7% w/w; or about 8% w/w. Most preferably 1.5% w/w to 6.0% w/w.

In some embodiments, the particulate co-processed pre-formulated excipient composition includes about 0.1% w/w to 5.0% w/w solubility enhancer; In some embodiments about 0.1% w/w; about 0.5% w/w; about 1% w/w; about 1.5% w/w; about 2% w/w; about 2.5% w/w; about 3% w/w; about 3.5% w/w; about 4% w/w; about 4.5% w/w; or about 5% w/w. Most preferably 0.1% w/w to 1% w/w.

In some embodiments, the particulate co-processed pre-formulated excipient composition includes about 0.1% w/w to 5.0% w/w lubricant; In some embodiments about 0.1% w/w; about 0.5% w/w; about 1% w/w; about 1.5% w/w; about 2% w/w; about 2.5% w/w; about 3% w/w; about 3.5% w/w; about 4% w/w; about 4.5% w/w; or about 5% w/w. Most preferably 0.1% w/w to 2% w/w.

In an embodiment, the present invention provides a particulate co-processed pre-formulated excipient composition comprises based on total weight of the composition:
filler-binder in an amount of 88.0% w/w to 97.8% w/w;
glidant in an amount of 0.50% w/w to 3.0% w/w;
disintegrant in an amount of 1.5% w/w to 6.0% w/w;
solubility enhancer in an amount of 0.1% w/w to 1.0% w/w; and
lubricant in an amount of 0.1% w/w to 2.0% w/w.

In a preferred embodiment of the present invention, the particulate co-processed pre-formulated excipient composition comprises MCC as filler-binder, colloidal silicon dioxide as glidant, sodium lauryl sulphate as solubility enhancer, croscarmellose sodium as disintegrant as and magnesium stearate as lubricant.

In an embodiment, the present invention provides a particulate co-processed pre-formulated excipient composition comprises based on total weight of the composition:
MCC in an amount of 88.0% w/w to 97.8% w/w;
Colloidal silicon dioxide in an amount of 0.50% w/w to 3.0% w/w;
croscarmellose sodium in an amount of 1.5% w/w to 6.0% w/w;
sodium lauryl sulphate in an amount of 0.1% w/w to 1.0% w/w; and
magnesium stearate in an amount of 0.1% w/w to 2.0% w/w.

In an embodiment of the present invention, pH of the particulate co-processed pre-formulated excipient composition ranges between 5.0 and 7.5.

In an embodiment of the present invention, bulk density of the particulates of the particulate co-processed pre-formulated excipient composition ranges between 0.20 g/ml to 0.65 g/ml. Most preferably 0.30 g/ml to 0.65 g/ml.

In an embodiment of the present invention, moisture content of the particulates of the particulate co-processed pre-formulated excipient composition is below 7% and ranges between 4% and 6%. Most preferably 4% to 5%.

In an embodiment of the present invention, particulates of the particulate co-processed pre-formulated excipient composition has median particle size distribution ranging in the micrometer scale. However, many microparticles have wider ranges of sizes. In some embodiments, the microparticles may have a diameter of at least about 1 μm, 10 μm, 50 μm, 100 μm, 200 μm, 300 μm, 400 μm, 500 μm, 600 μm, 700 μm, 800 μm, 900 μm, or 1000 μm. In some embodiments, the microparticles may have a diameter of less than 1000 μm, 900 μm, 800 μm, 700 μm, 600 μm, 500 μm, 250 μm, or less than 100 μm. The diameter of microparticles can range from any of the minimum values described above to any of the maximum values described above, for example from 1 μm to 1000 μm, 50 μm to 500 μm, 10 μm to 250 μm, 20 μm to 200 μm, or 50 μm to 100 μm. Preferably, the size of the microparticles ranges from about 25 μm to about 250 μm. Most preferably, the size of the microparticles ranges from about 50 μm to about 250 μm.

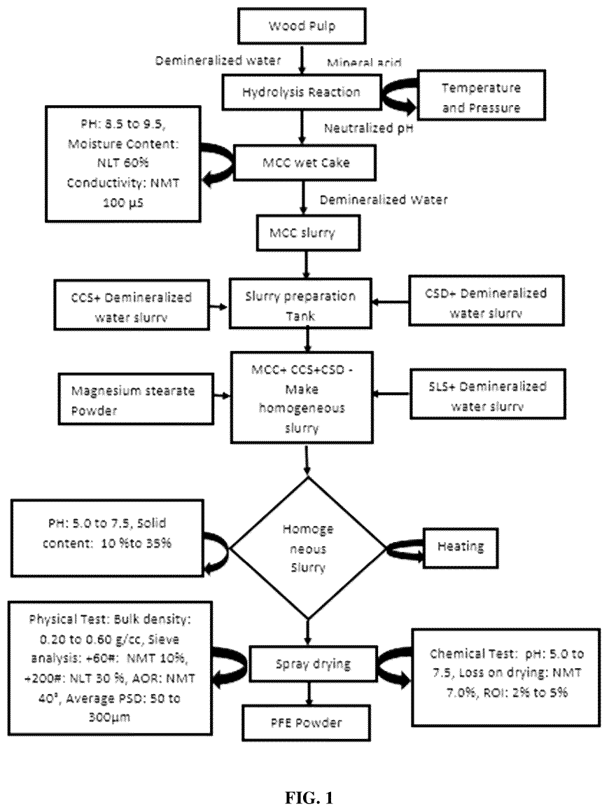

In an embodiment, the present invention provides a method for preparing the particulate co-processed pre-formulated excipient composition (FIG. 1), the method comprising the steps of:
a. forming a homogeneous slurry of filler-binder using demineralized water;
b. forming a homogeneous slurry of glidant using demineralized water;
c. forming a homogeneous slurry of disintegrant using demineralized water;
d. mixing the individual homogenous slurries of filler-binder, glidant and disintegrant by stirring to obtain a homogenous mixed slurry;
e. mixing a lubricant into the homogenous mixed slurry from step d), followed by proper mixing by stirring;
f. forming a homogeneous slurry of solubility enhancer using demineralized water;
g. adding the homogeneous slurry of solubility enhancer into the homogenous mixed slurry from step e), followed by proper mixing to obtain a slurry of co-processed pre-formulated excipient composition; and
h. spray or flash drying the slurry of co-processed pre-formulated excipient composition to obtain the particulate co-processed pre-formulated excipient composition; and
   wherein, the mixing is effected by agitation for 5 minutes at 25 rpm;
   wherein, temperature during mixing is maintained at 30° C. to 150° C.; and
   wherein, pH during mixing is maintained at 5 to 7.5.

In an embodiment, the present invention provides a method for preparing the particulate co-processed pre-formulated excipient composition (FIG. 1), the method comprising the steps of:
a. forming a homogeneous slurry of 88.0% w/w to 97.8% w/w MCC using demineralized water;
b. forming a homogeneous slurry of 0.50% w/w to 3.0% w/w colloidal silicon dioxide using demineralized water;
c. forming a homogeneous slurry of 1.5% w/w to 6.0% w/w croscarmellose sodium using demineralized water;
d. mixing the individual homogenous slurries of MCC, colloidal silicon dioxide and croscarmellose sodium by stirring to obtain a homogenous mixed slurry;
e. mixing 0.1% w/w to 2.0% w/w magnesium stearate powder into the homogenous mixed slurry from step d), followed by proper mixing by stirring;
f. forming a homogeneous slurry of 0.1% w/w to 1.0% w/w sodium lauryl sulphate using demineralized water;
g. adding the homogeneous slurry of 0.1% w/w to 1.0% w/w sodium lauryl sulphate into the homogenous mixed slurry from step e), followed by proper mixing to obtain a slurry of co-processed pre-formulated excipient composition; and
h. spray or flash drying the slurry of co-processed pre-formulated excipient composition to obtain the particulate co-processed pre-formulated excipient composition;
   wherein, the mixing is effected by agitation for 5 minutes at 25 rpm;
   wherein, temperature during mixing is maintained at 30° C. to 150° C.; and
   wherein, pH during mixing is maintained at 5 to 7.5.

In an embodiment of the present invention, all the individual excipients i.e. croscarmellose sodium, colloidal silicon dioxide, magnesium stearate, and Sodium lauryl sulphate are together coated on each of the Microcrystalline cellulose particles homogeneously, which after drying results in the particulate co-processed pre-formulated excipient composition.

In another embodiment of the present invention, solid content of the homogenous slurry is less than 50%.

In an embodiment of the present invention, the particulate co-processed pre-formulated excipient composition may further comprise one or more excipients selected from the group consisting of diluents, bulking agents, vehicles, pH adjusting agents, anti-oxidants, buffers, antiadherants, coating agents, preservatives, emulsifiers, suspending agents, release controlling agents, polymers, colorants, flavoring agents, plasticizers, solvents, preservatives, glidants, and chelating agents; used either alone or in combination.

In an embodiment of the present invention, the term “oral solid dosage formulation” shall be construed to include a particulate co-processed pre-formulated excipient composition plus an API, and optionally one or more excipients.

In an embodiment of the present invention, the oral solid dosage formulations are generally administered orally to patients, which include, but are not limited to, mammals, for example, humans, in the form of, for example, a tablet, a caplet, pills, capsules, granules or a suspension.

In an embodiment of the present invention, the oral solid dosage formulations are pharmaceutical, herbal, ayurvedic, or nutraceutical formulations.

In a preferred embodiment, the present invention provides a tablet formulation comprising:
the particulate co-processed pre-formulated excipient composition in an amount ranging between 35% w/w and 65% w/w;
a poorly soluble active pharmaceutical ingredient in an amount ranging between 35% w/w and 65% w/w; and
optionally one or more excipients in an amount ranging between 1% w/w and 10% w/w.

In an embodiment of the present invention, the tablet may be prepared by wet granulation, direct compression and/or dry granulation methods

In another aspect, the present invention provides a particulate co-processed pre-formulated excipient composition suitable for tableting by wet granulation, direct compression and/or dry granulation.

In another embodiment of the present invention, the particulate co-processed pre-formulated excipient composition that enhances final product tablet quality.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Example: 1 Particulate Co-Processed Pre-Formulated Excipient Composition 1

TABLE 1
Particulate co-processed pre-formulated excipient composition 1

| | Composition | |
|---|---|---|
| | MCC: 90.8, CSD: 3.0, | MCC: 97.6, CSD: 0.5, |
| | CCS: 6.0, SLS: 0.1, | CCS: 1.5, SLS, 0.2, |
| | Magnesium stearate 0.1 | Magnesium stearate 0.2 |
| Characteristics | Result | Result |
| Bulk density g/ml | 0.55 | 0.20 |
| Loss on drying % | 4.2 | 7.00 |
| Angle of Repose ° | 25 | 45 |
| Average Particle | 102 | 100 |
| size μm | | |

Tablet Profile

| | | |
|---|---|---|
| Tablet weight (mg) | 398 | 399 |
| Hardness (N) | 59 | 65 |
| Thickness (mm) | 3.2 | 3.9 |

### Example: 2 Particulate Co-Processed Pre-Formulated Excipient Composition 2

TABLE 2
Particulate co-processed pre-formulated excipient composition 2

| | Composition | |
|---|---|---|
| | MCC: 97.3, CSD: 0.6, | MCC: 96.4, CSD: 0.8, |
| | CCS: 1.5, SLS: 0.3, | CCS: 2.0, SLS: 0.4, |
| | Magnesium stearate 0.3 | Magnesium stearate 0.4 |
| Characteristics | Result | Result |
| Bulk density g/ml | 0.22 | 0.24 |
| Loss on drying % | 4.2 | 4.3 |
| Angle of Repose ° | 43 | 42 |
| Average Particle | 106 | 102 |
| size μm | | |

Tablet Profile

| | | |
|---|---|---|
| Tablet weight (mg) | 397 | 398 |
| Hardness (N) | 63 | 64 |
| Thickness (mm) | 3.8 | 3.8 |

### Example: 3 Particulate Co-Processed Pre-Formulated Excipient Composition 3

TABLE 3
Particulate co-processed pre-formulated excipient composition 3

| | Composition | |
|---|---|---|
| | MCC: 96.0, CSD: 1.0, | MCC: 95.1, CSD: 1.2, |
| | CCS: 2.0, SLS: 0.5, | CCS: 2.5, SLS: 0.6, |
| | Magnesium stearate 0.5 | Magnesium stearate 0.6 |
| Characteristics | Result | Result |
| Bulk density g/ml | 0.26 | 0.30 |
| Loss on drying % | 4.0 | 3.9 |
| Angle of Repose ° | 40 | 39 |
| Average Particle | 99 | 97 |
| size μm | | |

Tablet Profile

| | | |
|---|---|---|
| Tablet weight (mg) | 400 | 401 |
| Hardness (N) | 72 | 72 |
| Thickness (mm) | 3.7 | 3.7 |

### Example: 4 Particulate Co-Processed Pre-Formulated Excipient Composition 4

TABLE 4
Particulate co-processed pre-formulated excipient composition 4

| | Composition | |
|---|---|---|
| | MCC: 94.6, CSD: 1.4, | MCC: 93.6, CSD: 1.6, |
| | CCS: 2.5, SLS: 0.7, | CCS: 3.0, SLS: 0.8, |
| | Magnesium stearate: 0.8 | Magnesium stearate: 1.0 |
| Characteristics | Result | Result |
| Bulk density g/ml | 0.33 | 0.35 |
| Loss on drying % | 4.3 | 4.0 |
| Angle of Repose ° | 38 | 37 |
| Average Particle | 99 | 102 |
| size μm | | |

Tablet Profile

| | | |
|---|---|---|
| Tablet weight (mg) | 399 | 402 |
| Hardness (N) | 75 | 78 |
| Thickness (mm) | 3.6 | 3.5 |

### Example: 5 Particulate Co-Processed Pre-Formulated Excipient Composition 5

TABLE 5
Particulate co-processed pre-formulated excipient composition 5

| | Composition | |
|---|---|---|
| | MCC: 92.6, CSD: 1.8, | MCC: 91.6, CSD: 2.0, |
| | CCS: 3.5, SLS: 0.9, | CCS: 4.0, SLS: 1.0, |
| | Magnesium stearate: 1.2 | Magnesium stearate: 1.4 |
| Characteristics | Result | Result |
| Bulk density g/ml | 0.38 | 0.40 |
| Loss on drying % | 4.2 | 4.0 |
| Angle of Repose ° | 36 | 33 |
| Average Particle | 108 | 110 |
| size μm | | |

Tablet Profile

| | | |
|---|---|---|
| Tablet weight (mg) | 398 | 401 |
| Hardness (N) | 90 | 85 |
| Thickness (mm) | 3.4 | 3.5 |

### Example: 6 Particulate Co-Processed Pre-Formulated Excipient Composition 6

TABLE 6
Particulate co-processed pre-formulated excipient composition 6

| | Composition | |
|---|---|---|
| | MCC: 90.9, CSD: 2.5, | MCC: 89.9, CSD: 3.0, |
| | CCS: 4.0, SLS: 1.0, | CCS: 4.5, SLS: 0.8, |
| | Magnesium stearate: 1.6 | Magnesium stearate: 1.8 |
| Characteristics | Result | Result |
| Bulk density g/ml | 0.45 | 0.47 |
| Loss on drying % | 4.0 | 4.0 |
| Angle of Repose ° | 33 | 33 |
| Average Particle | 110 | 115 |
| size μm | | |

Tablet Profile

| | | |
|---|---|---|
| Tablet weight (mg) | 399 | 400 |
| Hardness (N) | 102 | 108 |
| Thickness (mm) | 3.3 | 3.2 |

### Example: 7 Particulate Co-Processed Pre-Formulated Excipient Composition 7

TABLE 7
Particulate co-processed pre-formulated excipient composition 7

| | Composition | |
|---|---|---|
| | MCC: 89.7, CSD: 2.5, | MCC: 89.7, CSD: 2.0, |
| | CCS: 5.0, SLS: 0.8, | CCS: 5.5, SLS: 0.8, |
| | Magnesium stearate: 2.0 | Magnesium stearate: 2.0 |
| Characteristics | Result | Result |
| Bulk density g/ml | 0.48 | 0.49 |
| Loss on drying % | 4.0 | 4.2 |
| Angle of Repose ° | 32 | 32 |
| Average Particle | 120 | 120 |
| size μm | | |

Tablet Profile

| | | |
|---|---|---|
| Tablet weight (mg) | 400 | 402 |
| Hardness (N) | 103 | 108 |
| Thickness (mm) | 3.3 | 3.3 |

### Example: 8 Particulate Co-Processed Pre-Formulated Excipient Composition 8

TABLE 8
Particulate co-processed pre-formulated excipient composition 8

| | Composition | |
|---|---|---|
| | MCC: 88.0, CSD: 3.0, | MCC: 90.0, CSD: 2.0, |
| | CCS: 6.0, SLS: 0.8, | CCS: 6.0, SLS: 0.5, |
| | Magnesium stearate: 2.0 | Magnesium stearate 1.5 |
| Characteristics | Result | Result |
| Bulk density g/ml | 0.49 | 0.50 |
| Loss on drying % | 4.2 | 4.0 |
| Angle of Repose ° | 32 | 31 |
| Average Particle | 120 | 123 |
| size μm | | |

Tablet Profile

| | | |
|---|---|---|
| Tablet Weight(mg) | 400 | 401 |
| Hardness (N) | 99.4 | 103 |
| Thickness (mm) | 3.7 | 3.8 |

### Example: 9 Particulate Co-Processed Pre-Formulated Excipient Composition 9

TABLE 9
Particulate co-processed pre-formulated excipient composition 9

| | Composition | |
|---|---|---|
| | MCC: 92.5, CSD: 2.0, | MCC: 92.4, CSD: 1.9, |
| | CCS: 4.5 SLS: 0.5, | CCS: 4.7, SLS: 0.5, |
| | Magnesium stearate: 0.5 | Magnesium stearate: 0.5 |
| Characteristics | Result | Result |
| Bulk density g/ml | 0.42 | 0.44 |
| Loss on drying % | 3.8 | 4.5 |
| Angle of Repose ° | 30 | 33 |
| Average Particle | 112 | 100 |
| size μm | | |

Tablet Profile

| | | |
|---|---|---|
| Tablet Weight (mg) | 400 | 402 |
| Hardness (N) | 174 | 172 |
| Thickness (mm) | 3.5 | 3.5 |

### Example 10: Tablet In-Vitro Evaluation and Release Profile of Hydrochlorothiazide Active Using Physical Blend and Co-Processed Pre-Formulated Excipient Composition Containing Tablet

TABLE 10
Tablet in-vitro evaluation of Hydrochlorothiazide active
using physical blend and co-processed pre-formulated
excipient composition containing Tablet
Tablet Evaluation

| | | | Co-processed |
|---|---|---|---|
| | | Physical blend | PFE |
| | Without SLS | MCC: 92.5, CSD: | MCC: 92.5, CSD: |
| | MCC: 93, CSD: | 2.0, CCS: 4.5, | 2.0, CCS: 4.5, |
| | 2.0, CCS: 4.5, | SLS: 0.5, | SLS: 0.5, |
| | Magnesium | Magnesium | Magnesium |
| Characteristics | stearate: 0.5 | stearate: 0.5 | stearate: 0.5 |
| Tablet Average | 102 | 100 | 100 |
| weight (mg) | | | |
| Hardness (N) | 113 | 114 | 121 |
| Thickness(mm) | 3.00 | 2.94 | 3.00 |
| Disintegration | 28 | 52 | 40 |
| Time (Sec) | | | |
| Friability (%) | 0.33 | 0.205 | 0.190 |

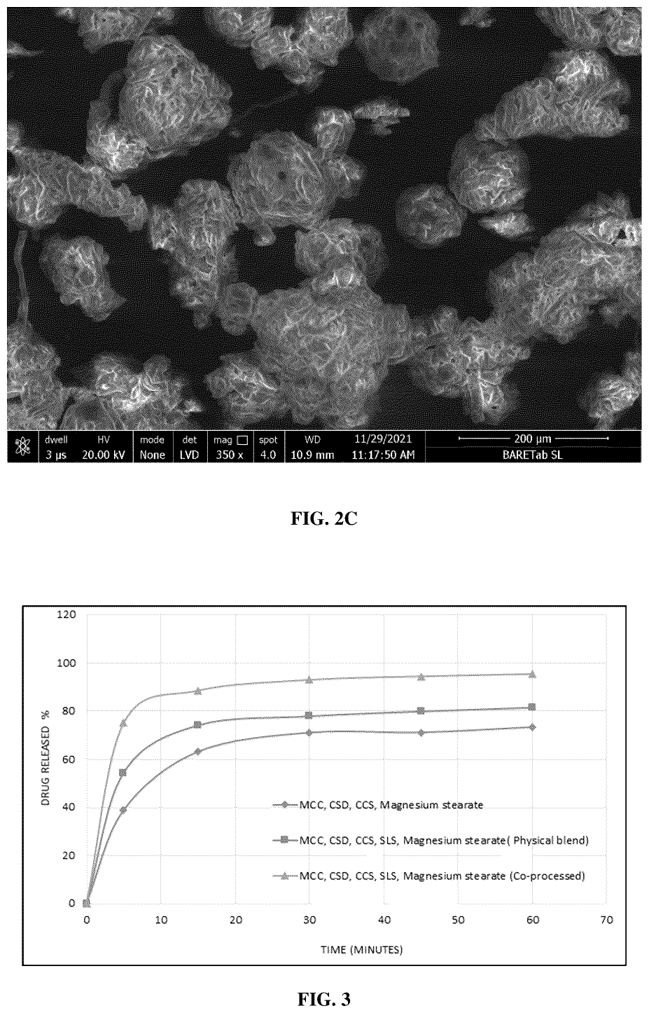

TABLE 11
Tablet Release profile of Hydrochlorothiazide active using physical blend
and co-processed pre-formulated excipient composition containing Tablet
API released profile %

| | API released Time (Minutes) (FIG. 3) | | |
|---|---|---|---|
| | Without SLS | Physical blend | Co-processed PFE |
| | MCC: 93, CSD: | MCC: 92.5, CSD: | MCC: 92.5, CSD: |
| | 2.0, CCS: 4.5, | 2.0, CCS: 4.5, SLS: | 2.0, CCS: 4.5, SLS: |
| | Magnesium | 0.5, Magnesium | 0.5, Magnesium |
| | stearate: 0.5 | stearate: 0.5 | stearate: 0.5 |
| | Hydrochlorothiazide 25 mg tablet composition | | |
| | API: 25% and | | |
| | Without SLS blend | API: 25% and | API: 25% and Co- |
| | Excipient blend | Excipient Physical | processed PFE |
| | 75% | blend 75% | 75% |
| 5 | 38.75 | 54.33 | 74.97 |
| 15 | 63.08 | 73.97 | 88.43 |
| 30 | 70.98 | 77.71 | 92.85 |
| 45 | 71.05 | 79.69 | 94.30 |
| 60 | 73.28 | 81.32 | 95.28 |

### Example 11: Morphological Analysis of Co-Processed Pre-Formulated Excipient Composition 9

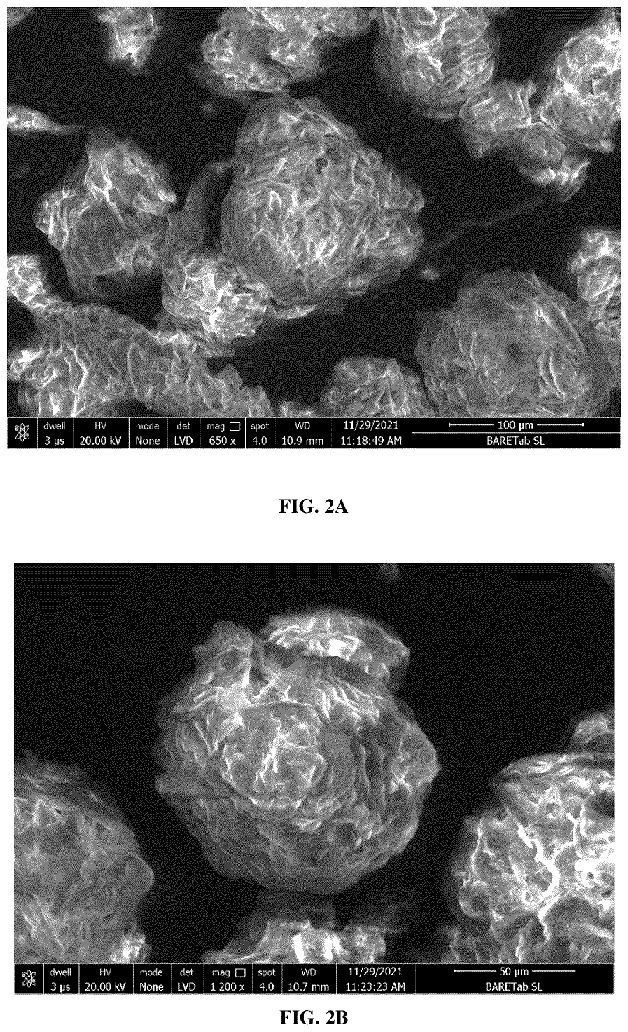

The composition 9 comprising MCC: 92.5, CSD: 2.0, CCS: 4.5, SLS: 0.5, Magnesium stearate: 0.5 analysed by the Scanning electron microscopy (SEM). FIG. 2A-2C represent the SEM images of the particles of composition 9.

Various modification and variation of the described assays, techniques and various means disclosed herein to implement the assays/methods in accordance with the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A particulate co-processed pre-formulated excipient composition for poorly soluble active pharmaceutical ingredients, said excipient composition comprises based on total weight of the composition:
filler-binder in an amount of 88.0% w/w to 97.8% w/w;
glidant in an amount of 0.50% w/w to 3.0% w/w;
disintegrant in an amount of 1.5% w/w to 6.0% w/w;
solubility enhancer in an amount of 0.1% w/w to 1.0% w/w; and
lubricant in an amount of 0.1% w/w to 2.0% w/w,
wherein, said excipient composition enhances dissolution rate of poorly soluble pharmaceutical active ingredient in oral solid dosage forms,
wherein, said excipient composition is ready-to-use composition.

2. The excipient composition as claimed in claim 1, wherein microcrystalline cellulose (MCC) used as filler-binder, colloidal silicon dioxide used as glidant, sodium lauryl sulphate used as solubility enhancer, croscarmellose sodium used as disintegrant and magnesium stearate used as lubricant.

3. The excipient composition as claimed in claim 1, wherein the particulates have an average particle size of 50 μm to 250 μm.

4. The excipient composition as claimed in claim 1, wherein pH of the excipient composition is 5.0-7.5.

5. The excipient composition as claimed in claim 1, wherein moisture content of the excipient composition is below 7%.

6. The excipient composition as claimed in claim 1, wherein the bulk density of the particulates is 0.30 g/ml to 0.65 g/ml.

7. The excipient composition as claimed in claim 1, wherein the oral solid dosage formulation is a tablet, a pill, or a capsule.

8. A method for preparing the particulate co-processed pre-formulated excipient composition as claimed in claim 1, the method comprising the steps of:
a. forming a homogeneous slurry of filler-binder using demineralized water;
b. forming a homogeneous slurry of glidant using demineralized water;
c. forming a homogeneous slurry of disintegrant using demineralized water;
d. mixing the individual homogenous slurries of filler-binder, glidant and disintegrant by stirring to obtain a homogenous mixed slurry;
e. mixing a lubricant into the homogenous mixed slurry from step d), followed by proper mixing by stirring;
f. forming a homogeneous slurry of solubility enhancer using demineralized water;
g. adding the homogeneous slurry of solubility enhancer into the homogenous mixed slurry from step e), followed by proper mixing to obtain a slurry of co-processed pre-formulated excipient composition; and
h. spray or flash drying the slurry of co-processed pre-formulated excipient composition to obtain the particulate co-processed pre-formulated excipient composition; and
wherein, the mixing is effected by agitation for 5 minutes at 25 rpm;
wherein, temperature during mixing is maintained at 30° C. to 150° C.;
wherein, pH during mixing is maintained at 5 to 7.5; and
wherein, loss on drying of particulates is 4% to 5%.

9. The method as claimed in claim 9, wherein microcrystalline cellulose (MCC) used as filler-binder, colloidal silicon dioxide used as glidant, sodium lauryl sulphate used as solubility enhancer, croscarmellose sodium used as disintegrant and magnesium stearate used as lubricant.

10. The method as claimed in claims 9-10, the method comprising the steps of:
a. forming a homogeneous slurry of 88.0% w/w to 97.8% w/w MCC using demineralized water;
b. forming a homogeneous slurry of 0.50% w/w to 3.0% w/w colloidal silicon dioxide using demineralized water;
c. forming a homogeneous slurry of 1.5% w/w to 6.0% w/w croscarmellose sodium using demineralized water;
d. mixing the individual homogenous slurries of MCC, colloidal silicon dioxide and croscarmellose sodium by stirring to obtain a homogenous mixed slurry;
e. mixing 0.1% w/w to 2.0% w/w magnesium stearate powder into the homogenous mixed slurry from step d), followed by proper mixing by stirring;
f. forming a homogeneous slurry of 0.1% w/w to 1.0% w/w sodium lauryl sulphate using demineralized water;
g. adding the homogeneous slurry of 0.1% w/w to 1.0% w/w sodium lauryl sulphate into the homogenous mixed slurry from step e), followed by proper mixing to obtain a slurry of co-processed pre-formulated excipient composition; and
h. spray or flash drying the slurry of co-processed pre-formulated excipient composition to obtain the particulate co-processed pre-formulated excipient composition;
wherein, the mixing is effected by agitation for 5 minutes at 25 rpm;
wherein, temperature during mixing is maintained at 30° C. to 150° C.;
wherein, pH during mixing is maintained at 5 to 7.5; and
wherein, loss on drying of particulates is 1.5% to 5%.

11. A tablet formulation comprising:
the particulate co-processed pre-formulated excipient composition as claimed in claim 1 in an amount ranging between 35% w/w and 65% w/w;
a poorly soluble active pharmaceutical ingredient in an amount ranging between 35% w/w and 65% w/w; and
optionally one or more excipients in an amount ranging between 1% w/w and 10% w/w.

12. The tablet formulation as claimed in claim 12, wherein the one or more excipients is selected from the group consisting of diluents, disintegrants, fillers, bulking agents, vehicles, pH adjusting agents, stabilizers, anti-oxidants, binders, buffers, lubricants, antiadherants, coating agents, preservatives, emulsifiers, suspending agents, release controlling agents, polymers, colorants, flavoring agents, plasticizers, solvents, preservatives, glidants, and chelating agents; used either alone or in combination.

13. The tablet formulation as claimed in claim 12, wherein the tablet is prepared by wet granulation, direct compression and/or dry granulation methods.
